(19) United States
(12) Patent Application Publication
(10) Pub. No.: US 2005/0245794 A1
(43) Pub. Date: Nov. 3, 2005

(54) Communication with implantable monitoring probe

(75) Inventor: David A. Dinsmoor, St. Paul, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/835,424

(22) Filed: Apr. 29, 2004

(51) Int. Cl. A61B 5/07; A61B 5/00; A61B 7/00; A61B 5/117; A61B 5/103

(52) U.S. Cl. 600/302; 600/549; 600/561; 600/309

(57) ABSTRACT

Devices and techniques are disclosed for sensing a physiological parameter, such as a physiological parameter proximate to the gastrointestinal tract of a patient. A transducer generates an analog electric signal as a function of the sensed physiological parameter. In some embodiments, the analog signal is sent to an analog communication system for transmission to an external receiver. Some embodiments support storing the analog signal in analog form.

### TECHNICAL FIELD

The present invention relates to implantable physiological monitoring systems, and more particularly to implantable probes for monitoring one or more parameters in the body of a patient.

### BACKGROUND

A patient can benefit from monitoring of physiological parameters proximate to his gastrointestinal tract. A patient suffering from gastroesophageal reflux, for example, suffers from a condition in which gastric acid refluxes, or flows in the direction opposite to the normal flow, from the stomach into the esophagus. Frequent reflux episodes may result in a potentially severe problem known as gastroesophageal reflux disease, which is a common cause of dyspepsia or heartburn. As a common cause of chest pain, gastroesophageal reflux disease frequently mimics the symptoms of a myocardial infarction or severe angina pectoris, which are signs of severe coronary artery disease. Because their treatments and outcomes are different, distinguishing between gastroesophageal reflux disease and coronary artery disease is of diagnostic importance to the patient and physician.

To monitor conditions such as gastroesophageal reflux disease, a compact monitoring device can be deployed at an implantation site in a body of a patient. The monitoring device can be deployed proximate to the lower esophageal sphincter, for example, and can monitor the pH of fluids proximate to the lower esophageal sphincter.

In a similar fashion, one or more compact monitoring devices can be deployed at other sites along the gastrointestinal tract, and can monitor a variety of physiological parameters. Monitored physiological parameters can include pH, temperature or pressure. Monitored physiological parameters can also include ion concentration, such as the concentrations of sodium, potassium, calcium, magnesium, chloride, bicarbonate, or phosphate ions. Monitored physiological parameters can further comprise concentration of a solute within a body fluid, such as glucose, bilirubin, creatinine, blood urea nitrogen, urinary nitrogen, renin, and angiotensin.

Some of the monitoring devices are not configured for implantation in the body, but rather move through the gastrointestinal tract. In a conventional implantable monitor, data concerning a sensed physiological parameters is converted to a digital signal through an analog-to-digital converter. The data are thereby converted to digital form, and may be stored in a memory element in digital form. In some monitors, the digital data are transmitted out of the body of the patient via digital communication systems.

Table 1 below lists documents that disclose various techniques for monitoring physiological parameters proximate to the gastrointestinal tract and transmitting signals reflecting the sensed parameters out of the body.

TABLE 1

| Patent Number | Inventors/Author | Title |
|---|---|---|
| 6,689,056 | Kilcoyne et al. | Implantable Monitoring Probe |
| 6,285,897 | Kilcoyne et al. | Remote physiological monitoring |
| | | system |
| 5,984,875 | Brune | Ingestible animal temperature sensor |
| 5,604,531 | Iddan et al. | In vivo video camera system |

All documents listed in Table 1 above are hereby incorporated by reference herein in their respective entireties. As those of ordinary skill in the art will appreciate readily upon reading the Summary of the Invention, Detailed Description of the Preferred Embodiments and claims set forth below, many of the devices and methods disclosed in the patents of Table 1 may be modified advantageously by using the techniques of the present invention.

### SUMMARY

In general, the invention is directed to devices and techniques that move information about monitored physiological parameters from a monitoring device proximate to the gastrointestinal tract of a patient to an external device, i.e., a device outside the body of the patient. In particular, the invention is directed to techniques of data handling, storage and encoding for transmission via radio frequency transmission.

In contrast to conventional probes deployed along the gastrointestinal tract that process and transmit information in digital form, the invention is directed to probes deployed along the gastrointestinal tract that transmit analog information. The data representative of the monitored physiological parameters are not digitized, but rather are stored in analog form, are transmitted in analog form, or both.

Various embodiments of the present invention provide solutions to one or more problems existing in the prior art with respect to prior techniques for handling signals that reflect physiological parameters and for transmitting the signals outside the body of the patient. These problems include the need for circuitry to convert analog signals to digital signals and to use the digital signals to modulate a communication system. In addition, monitoring devices deployed proximate to the gastrointestinal tract may suffer from undesirable size and power consumption due to the presence of digital electronics.

Various embodiments of the present invention are capable of solving at least one of the foregoing problems in the existing art. When embodied in an implantable monitoring device, the invention includes features that improve efficiency, eliminate electronic components, and consequently save power and space. In some embodiments of the invention, a monitoring device can be presented as a small capsule. The capsule can be implanted in the body of the patient proximate to the gastrointestinal tract by a variety of techniques. The implantation techniques vary in degree of invasiveness. Some techniques, such as implantation with an endoscope, are not very invasive. In addition, the implantation techniques vary in degree of duration of implantation. Some implantations, such as those that anchor the monitoring device with a dissolving suture, terminate on their own within a few days. Other implantations last for longer durations.

In some embodiments, a transducer generates an analog electrical signal as a function of the sensed physiological parameter, and an analog communication system transmits the electrical signal to an external receiver. An analog communication system modulates an analog communication signal with the analog electrical signal. Some of the embodiments use analog communication signal encoding techniques such as phase modulation or frequency modulation. In this way, the monitoring device can provide “real time” transmission of data that reflect one or more sensed physiological parameters.

In other embodiments, the monitoring device stores the analog electrical signal for later transmission. To avoid conversion of the analog signal to a digital signal, the analog electrical signal can be stored in an analog memory circuit.

In comparison to known techniques for placement of medical devices within the esophagus, or elsewhere in the gastrointestinal tract, various embodiments of the invention may provide one or more advantages. For example, various embodiments of the invention prevent loss of information that can result when an analog electrical signal is converted to a digital signal. Various embodiments of the invention also save power and space by avoiding analog-to-digital conversion.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic diagram illustrating a monitoring device implanted proximate to a gastrointestinal tract.

FIG. 2 is a functional block diagram illustrating a monitoring device configured to be implanted proximate to a gastrointestinal tract.

FIG. 3 is a flow diagram illustrating a method for generating, storing and transmitting analog electrical signals that reflect sensed physiological parameters.

FIG. 4 is a flow diagram illustrating a method for generating, storing and transmitting analog electrical signals that reflect sensed physiological parameters response to one or more instructions.

### DETAILED DESCRIPTION

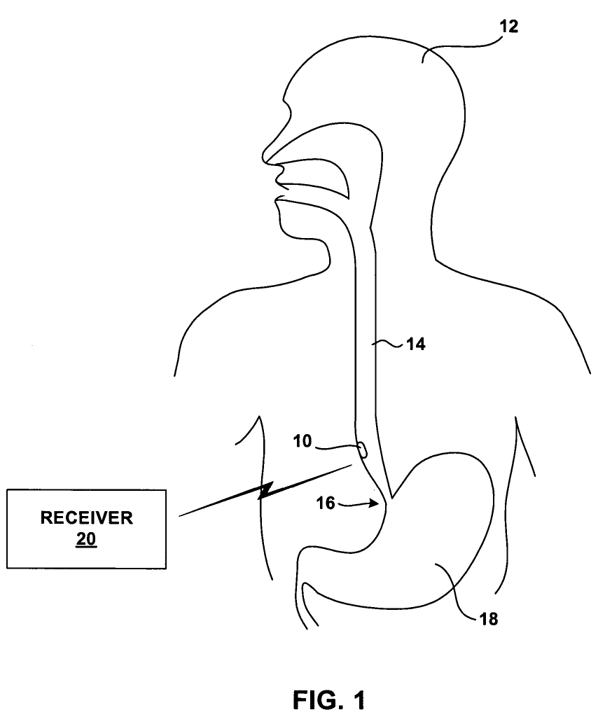

FIG. 1 is a schematic diagram illustrating a monitoring device 10 implanted proximate to a gastrointestinal tract of a patient 12. In the illustrated embodiment, monitoring device 10 is implanted in an esophagus 14 of patient 12, and can be configured to monitor any of several physiological parameters such as of pH, temperature, pressure, fluid flow, bolus passage, pressure and electrical activity. As shown in FIG. 1, monitoring device is sutured to the esophageal wall of a patient, approximately 5 centimeters above the lower esophageal sphincter 16. The invention is not limited to this deployment, however. Various monitoring devices may be deployed at numerous other sites proximate to the gastrointestinal tract. Monitoring devices may be introduced into the gastrointestinal tract of patient 12, or can be deployed along the gastrointestinal tract without actually being deployed in the gastrointestinal tract, e.g., by deployment beneath the mucosa of the gastrointestinal tract.

In this exemplary deployment, monitoring device 10 can monitor physiological parameters that allow a clinician to accurately diagnose gastroesophageal reflux disease. Monitoring device 10 can sense physiological parameters such as acidity and the direction of fluid flow, which may be important to a diagnosis of gastroesophageal reflux disease. Lower esophageal sphincter 16 normally relaxes to allow food to enter into stomach 18 from esophagus 14, and contracts to prevent stomach acids from entering esophagus 14. In patient 12 experiencing gastroesophageal reflux disease, lower esophageal sphincter 16 relaxes too frequently or at inappropriate times, allowing fluid to reflux from stomach 18 into the esophagus 14, which may lead to complications such as heartburn, painful swallowing, difficulty swallowing, coughing, wheezing, asthma, inflammation of the vocal cords or throat, esophageal ulcers, narrowing of the esophagus, and in the worst cases Barrett's esophagus.

As described below, monitoring device 10 includes one or more transducers that sense one or more physiological parameters and generate one or more analog electrical signals as a function of the sensed parameters. Monitoring device 10 further includes an analog communication system that transmits an analog wireless communication signal to a receiver 20 external to the body of patient 12. Receiver 20 is configured to demodulate the received analog communication signal and recover the analog signal or signals that reflect the sensed parameters. Receiver 20 can typically store the information received from monitoring device 10, and in some embodiments, can process the information. Receiver 20 may include a user interface, e.g., a keypad and display, and may display information received from monitoring device 10. In some embodiments of the invention, receiver 20 also transmits instructions to monitoring device 10. The invention is not limited to any particular embodiment of receiver 20, however.

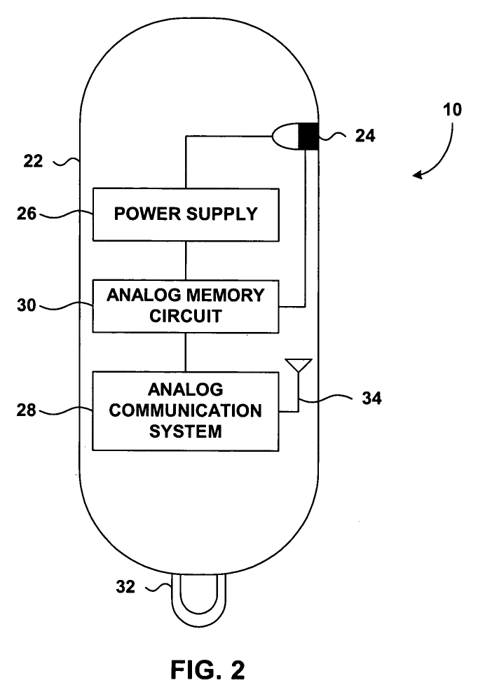

FIG. 2 is a schematic diagram illustrating an embodiment of an implantable medical monitoring device 10 according to one embodiment of the invention. Monitoring device 10 is configured to be deployed proximate to the GI tract of a patient. Monitoring device 10 includes an outer shell 22 that surrounds the electronic components of monitoring device 10. A transducer 24, power supply 26 and analog communication system 28 are encased within the outer shell 22. The embodiment of monitoring device 10 depicted in FIG. 1 also includes analog memory circuit 30.

In certain embodiments, the shape of shell 22 can resemble a capsule similar to that of a pill or gel capsule. Shell 22 can be made of any of various materials, including plastics such as polycarbonates, polyethylene, polytetrafluoroethelyne, nylon, delrin, or polyethylene terephthalate. The material used for shell 22 should ordinarily be resistant to water and acidic environments, as shell can be deployed, in some embodiments, to food, water, and gastrointestinal contents, including highly caustic gastric acid. In a typical implementation, a capsule-shaped shell 22 could be about two and half centimeters long, and about 0.7 centimeters in diameter, although the invention is not limited to these dimensions.

Shell 22 can have a lubricious coating applied to its outer surface, which reduces friction between shell 22 and any object or material that comes in contact with the shell 22, such as an esophageal wall or any food or fluids that flow past monitoring device 10. Such a coating can be made of silicone, silicone derivatives, or other hydrophilic materials. The slippery coating can reduce the likelihood that material such as ingested material will adhere to monitoring device 10, can reduce the likelihood of tissue irritation, and can reduce the likelihood of dislodgement of monitoring device 10 due to passage of fluid or a solid bolus.

In the embodiment shown in FIG. 1, the capsule shape of shell 22 is streamlined with smooth rounded corners, which can help to avoid trauma to the gastrointestinal mucosa during endoscopic placement of monitoring device 10. The capsule shape also is advantageous when monitoring device 10 becomes unattached from its implantation site and passes through the gastrointestinal tract and is excreted in the stool. In some deployments, monitoring device 10 is implanted non-permanently, an is expected to detach from the implantation site after about two to ten days.

Monitoring device 10 can be implanted proximate to the gastrointestinal tract in a variety of ways. One deployment technique includes use of a flexible or rigid endoscope inserted through the nose or mouth of the patient. Monitoring device 10 can attached to a wall of the gastrointestinal tract in any number of ways, including a dissolvable suture that ties the mucosa to an attachment device such as eyelet 32. In another implementation, monitoring device 10 can anchored to a wall of the gastrointestinal tract with an anchoring device such as a dissolvable clip or by a biocompatible adhesive. Another deployment technique comprises implanting monitoring device 10 sub-mucosally. The invention is not limited to any particular implantation technique. Although the invention is suitable for temporary implantations in which monitoring device 10 loses attachment to the patient and passes from the body in the patient's stool, the invention supports implantations for long durations as well.

Examples of a monitoring device and implantation techniques are described in U.S. Pat. No. 6,689,056, which is incorporated herein by reference. The invention is not limited to the particular monitoring devices and implantation techniques described therein, however. As noted above, monitoring device 10 was described as sutured to the esophageal wall of patient 12, approximately 5 centimeters above lower esophageal sphincter 16, but this description was for purposes of illustrating a typical operation of monitoring device 10, and the invention is not limited to this deployment. Various monitoring devices may be deployed at numerous other sites proximate to the gastrointestinal tract.

Monitoring device 10 includes transducer 24, which includes a sensor configured to sense a physiological parameter and to generate an analog electrical signal as a function of the sensed physiological parameter. Transducer 24 can include, for example, a pH sensor that responds to acidity or alkalinity. For monitoring device 10 deployed proximate to the lower esophageal sphincter, a pH sensor is useful for monitoring gastroesophageal reflux disease.

Transducer 24 can also include a temperature sensor, a pressure sensor and a fluid flow sensor. Transducer 24 can also include an electrical activity sensor that responds to current, voltage, impedance or other electrical characteristic. Transducer 24 can further include a sensor configured to detect bolus passage, or a sensor configured to respond to one or more chemicals. In some embodiments, transducer 24 generates an analog electrical signal as a function of a sensed concentration of an ion, or a protein such as a hormone, or a chemical messenger such as a neurotransmitter. Transducer 24 can further respond to chemical substances such as glucose, bilirubin, creatinine, blood urea nitrogen, renin, blood oxygen and the like.

Transducer 24 may be powered by power supply 26, which may include a battery. In some embodiments of the invention, monitoring device 10 omits a battery, and is powered by another means externally via inductive energy transfer. The analog electrical signal generated as a function of the sensed physiological parameter may be processed by analog electronics such as an amplifier or filter (not shown) to improve signal quality and reduce noise. In the embodiment shown in FIG. 1, the analog electrical signal can be stored in analog memory circuit 30. Analog memory circuit 30 stores the analog signal from transducer 24 without an intervening conversion to digital data. Analog memory circuit 30 can store the analog signal generated by transducer 24 as a voltage value, for example. In the case of a time-varying signal, analog memory circuit 30 can store the time-varying analog signal as a series of voltage values in an array of capacitative elements, for example. Because analog to digital conversion is avoided, there is reduced demand upon power supply 26. Furthermore, some embodiments of the invention eliminate an analog-to-digital converter completely, thereby saving space. In addition, storage of analog data avoids data loss that can occur during conversion from analog to digital or vice-versa. An example of an analog memory circuit is described in U.S. Pat. No. 5,312,446, but the invention is not limited to the analog memory circuit described therein.

The analog signal generated by transducer 24, whether stored in analog memory circuit 30 or not, can be supplied to analog communication system 28. Analog communication system 28 transmits the analog electrical signal from inside a body of the patient to a receiver external to the body of the patient via an transmission element such as an antenna 34. In general, the analog signal modulates a signal generated by analog communication system 28, creating a analog communication signal. The external device (not shown) receives the analog communication signal and demodulates the analog communication signal to recover the analog signal that reflects the physiological parameter sensed by transducer 24. In some embodiments of the invention, analog communication system 28 includes a receiver that receives and demodulates messages received from an external unit, such as receiver 20. The received messages may include one or more instructions that direct monitoring device 10 to perform one or more operations.

Analog communication system 28 employs analog communication techniques rather than digital communication techniques. Analog communication system 28 receives the analog electrical signal, and uses the analog electrical signal to modulate the analog communication signal. Typically, analog communication system 28 employs angle modulation such as phase modulation (PM) or frequency modulation (FM), but the invention encompasses some embodiments that employ amplitude modulation (AM).

In some embodiments of the invention, a controller (not shown in FIG. 2) governs whether the analog signal generated by transducer 24 is transmitted immediately by analog communication system 28 or whether the analog signal generated by transducer 24 is stored in analog memory circuit 30 for later transmission. The controller, which can be embodied as a microprocessor, can perform one or more other functions, including regulating the times at which transducer 24 generates analog signals and responding to instructions received via analog communication system 28.

In FIG. 2, the elements of the invention are depicted for clarity as distinct elements. In some embodiments of the invention, however, the elements may be combined in a single physical element. For example, transducer 24 and analog communication system 28 may be embodied in a single physical component, or analog communication system 28 may be embodied in a single physical component with transmission element 34.

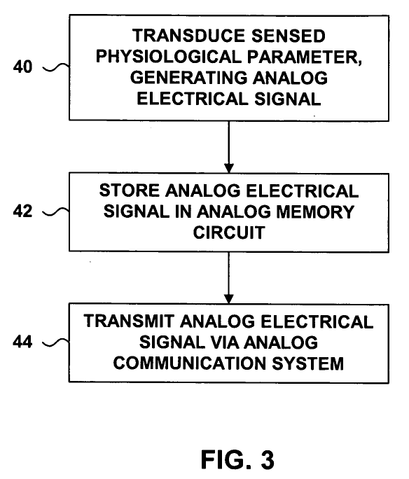

FIG. 3 is a flow diagram illustrating embodiments of the invention. Monitoring device 10 transduces a sensed physiological parameter via transducer 24, which senses the physiological parameter via a sensor located proximate to the gastrointestinal tract, and which generates an analog electrical signal as a function of the sensed physiological parameter (40). Optionally, monitoring device 10 stores the analog electrical signal in analog memory circuit 30 (42). Monitoring device 10 transmits the analog electrical signal from inside the body of the patient to an external receiver via analog communication system 28 (44).

In some embodiments of the invention, the analog electrical signal is transmitted as it is generated, and is not stored in analog memory circuit 30. In these embodiments, the analog electrical signal modulates an analog communication signal in “real time.”

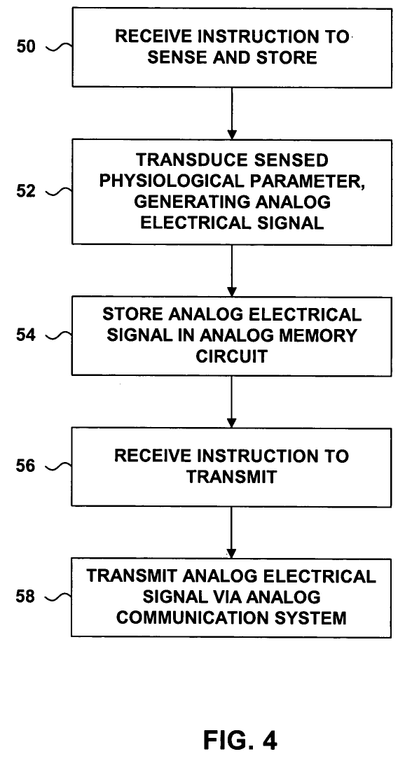

FIG. 4 is a flow diagram illustrating embodiments in which one or more operations are performed in response to an instruction from an external unit, such as receiver 20. An instruction is a signal that includes one or more directions to monitoring device 10.

In the exemplary method illustrated in FIG. 4, monitoring device 10 receives an instruction from an external unit to sense and store data pertaining to one or more physiological parameters (50). The instruction may specify, for example, which physiological parameters are to be monitored and how frequently the physiological parameters are to be monitored. Monitoring device 10 carries out sensing sand storing according to that the instructions (52, 54). When monitoring device 10 receives an interrogation instruction that directs monitoring device 10 to transmit the stored data (56), monitoring device 10 transmits according to that instruction (58).

The procedure depicted in FIG. 4 is for purposes of illustration, and many variations are within the scope of the invention. For example, an instruction may direct monitoring device 10 to begin sensing a physiological parameter and to commence transmitting the analog electrical signal via analog communication system 28, without storing the signal. An instruction may also interrogate monitoring device 10, directing monitoring device 10 to transmit a specific set of analog data stored in analog memory circuit 30 via analog communication system 28.

One of more advantages may result from keeping the electrical signals in analog form and transmitting them with an analog communication system. Many physiological parameters are, by their nature, analog parameters. Some digitizing techniques can result in loss of information when the analog electrical signal is converted to a digital signal. In addition, keeping the electrical signal in analog form can result in increased efficiency and power saving, by avoiding analog-to-digital conversion. Further, the techniques of the invention support the creation of monitoring devices that omit digital processing elements and are therefore compact, and that can be implanted by a variety of techniques.

Although some embodiments of the invention include no digital components, the invention encompasses embodiments that include digital components. As mentioned above, a controller, which can include one or more digital elements, can govern the operation of monitoring device 10. Further, some embodiments of the invention are well suited for deployment proximate to the gastrointestinal tract, but the invention includes some embodiments in which monitoring device 10 is not deployed proximate to the gastrointestinal tract.

The preceding specific embodiments are illustrative of the practice of the invention. It is to be understood, therefore, that other expedients known to those skilled in the art or disclosed herein may be employed without departing from the invention or the scope of the claims. For example, the invention need not be limited to capsule-shaped monitoring devices, or to monitoring devices that are configured to be deployed in the esophagus.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1. A method comprising:
transducing, via a sensor located proximate to a gastrointestinal tract of a patient, a sensed physiological parameter to an analog electrical signal; and
transmitting the analog electrical signal from inside a body of the patient to a receiver external to the body of the patient via an analog communication system,
wherein the analog communication system comprises a phase modulation communication system or a frequency modulation communication system.

2. The method of claim 1, wherein transducing the sensed physiological parameter to the analog electrical signal comprises generating the analog electrical signal as a function of one or more of pH, temperature, pressure, fluid flow, bolus passage, pressure or electrical activity.

3. The method of claim 1, wherein transducing the sensed physiological parameter to the analog electrical signal comprises generating the analog electrical signal as a function of a concentration of one or more of an ion or a protein.

4. The method of claim 1, wherein the sensed physiological parameter and the analog electrical signal are time-varying.

5. The method of claim 1, further comprising:
receiving an instruction from an external unit;
transducing the sensed physiological parameter to the analog electrical signal response to the instruction.

6. The method of claim 1, further comprising:
receiving an instruction from an external unit;
transmitting the analog electrical signal in response to the instruction.

7. A method comprising:
transducing, via a sensor located proximate to a gastrointestinal tract of a patient, a sensed physiological parameter to an analog electrical signal;
storing the analog electrical signal in an analog memory circuit; and
transmitting the analog electrical signal from inside a body of the patient to a receiver external to the body of the patient via an analog communication system.

8. The method of claim 1, wherein transducing the sensed physiological parameter to the analog electrical signal comprises generating the analog electrical signal as a function of one or more of pH, temperature, pressure, fluid flow, bolus passage, pressure or electrical activity.

9. The method of claim 7, wherein transducing the sensed physiological parameter to the analog electrical signal comprises generating the analog electrical signal as a function of a concentration of one or more of an ion or a protein.

10. The method of claim 7, wherein transducing the sensed physiological parameter comprises transducing the sensed physiological parameter at a first time, and wherein the analog electrical signal is a first analog electrical signal, the method further comprising:
transducing the sensed physiological parameter at a second time to a second analog electrical signal; and
storing the second analog electrical signal in the analog memory circuit.

11. The method of claim 10, wherein storing the first analog electrical signal in the analog memory circuit comprises storing a first voltage value, and wherein storing the second analog electrical signal in the analog memory circuit comprises storing a second voltage value.

12. The method of claim 7, wherein the analog communication system comprises one of an amplitude modulation communication system, a phase modulation communication system and a frequency modulation communication system.

13. A device comprising:
a transducer configured to sense a physiological parameter and to generate an analog electrical signal as a function of the sensed physiological parameter;
an analog communication system comprising a modulator configured to receive the analog electrical signal and to transmit an analog communication signal as a function of the analog electrical signal; and
a capsule-shaped shell sized for introduction into a gastrointestinal tract of a patient and configured to hold the transducer and the analog communication system,
wherein the analog communication system comprises a phase modulation communication system or a frequency modulation communication system.

14. The device of claim 13, further comprising an analog memory circuit configured to store the analog electrical signal.

15. The device of claim 13, wherein the transducer comprises a pH sensor, a temperature sensor, a pressure sensor, a fluid flow sensor, a pressure sensor or an electrical activity sensor.

16. A device comprising:
means for sensing a physiological parameter of a patient and generating an analog electrical signal as a function of the sensed physiological parameter;
means for receiving the analog electrical signal and for transmitting an analog communication signal as a function of the analog electrical signal via one of phase modulation and frequency modulation; and
means for holding the sensing means and the transmitting means proximate to the gastrointestinal tract of the patient.

17. The device of claim 16, further comprising means to store the analog electrical signal in analog form.

18. The device of claim 16, wherein the holding means comprises a capsule-shaped shell.

19. The device of claim 16, further comprising a means for attaching the holding means to a wall of the gastrointestinal tract.
